(19) United States
(12) Patent Application Publication
(10) Pub. No.: US 2013/0034899 A1
(43) Pub. Date: Feb. 7, 2013

(54) CELL CULTURING DEVICE USING ELECTRICAL RESPONSIVENESS FUNCTIONAL MATERIAL, CELL CULTURING SYSTEM INCLUDING THE SAME, AND CELL CULTURING METHOD

(71) Applicants: Ichiro Takemura, Tokyo (JP); Tomohiro Hayakawa, Saitama (JP); Eriko Matsui, Tokyo (JP)

(72) Inventors: Ichiro Takemura, Tokyo (JP); Tomohiro Hayakawa, Saitama (JP); Eriko Matsui, Tokyo (JP)

(73) Assignee: Sony Corporation, Tokyo (JP)

(21) Appl. No.: 13/552,335

(22) Filed: Jul. 18, 2012

(30) Foreign Application Priority Data
Aug. 3, 2011 (JP) 2011-169818

(51) Int. Cl. C12M 3/00; C12N 5/07; C12N 5/04

(52) U.S. Cl. 435/325; 435/289.1; 435/420

(57) ABSTRACT

A cell culturing device includes: an electrode on a surface of which a hydrophilicity/hydrophobicity converting material adapted to be electrically changed between hydrophilicity and hydrophobicity is provided. In this case, the electrode is disposed within a region in which a cell to be cultured is adapted to be accommodated. With the cell culturing device, the application of the suitable voltage to the electrode changes the hydrophilicity/the hydrophobicity of the hydrophilicity/hydrophobicity converting material. Thus, the feed material adsorbed to the hydrophilicity/hydrophobicity converting material is desorbed, thereby making it possible to feed the feed material to the cell.

### BACKGROUND

The present disclosure relates to a cell culturing device using an electrical responsiveness functional material, a cell culturing system including the same, and a cell culturing method using one of them. More specifically, the present disclosure relates to a cell culturing device utilizing a functional material which is electrically converted between hydrophilicity and hydrophobicity, a cell culturing system including the same, and a cell culturing method using one of them.

In general, in cell culture, a fetal calf serum is added to a culture solution. However, a performance difference for each lot is large, which causes the reproducibility of the experiments to be difficult. In addition, co-culture with a feeder cell such as a mouse embryonic fibroblast in the culture of a stem cell involves problems about contamination of a cell or tissue due to an exogenous infection factor, outbreak of rejection response due to a different source or species-derived component, and the like, which becomes a theme in proceeding to a clinical application. For this reason, in recent years, the study for replacement with a culture solution or an additive substance whose composition is chemically obvious in the cell culture has progressed.

In the case of the additive substance of the cell culturing solution described above, a suitable amount of additive substance needs to be added to the culture solution at suitable time. Then, Japanese Patent Laid-Open No. Hei 10-117767 discloses a cell culturing vessel to which a medical agent liquid solution can be introduced by a diaphragm which transmits a material having a low-molecular weight.

However, when the control for a component(s) of the cell culturing solution is more positively considered without relying on natural diffusion or the like of the material within the culture solution, there is considered a system for carrying out control due to some sort of stimulation from the outside. For example, Japanese Patent Laid-Open No. 2003-310244 discloses a technique with which in a cell culturing vessel using temperature responsiveness polymer, a change is caused in a temperature of a culture solution, thereby peeling off cells bonded to the cell culturing vessel.

### SUMMARY

The present disclosure has been made in order to solve the problems described above, and it is therefore desirable to provide a novel technique for cell culturing, in a word, a cell culturing device using an electrical responsiveness functional material which is capable of being electrically converted between hydrophilicity and hydrophobicity, a cell culturing system including the same, and a cell culturing method using one of them.

The replacement with the culture solution and the additive substance whose components are chemically obvious is important in the cell culture. However, a method of automatically supplying the material to the cell culturing solution with precise which is quantitatively and temporally high has not yet been proposed until now. Then, the inventors of this patent application have earnestly made the investigation and as a result, has found out a method in which a hydrophilicity/hydrophobicity converting material which is changed between the hydrophilicity and the hydrophobicity in accordance with a redox reaction is controlled by application of a suitable voltage to an electrode to desorb a hydrophilic or hydrophobic feed material adsorbed to the hydrophilicity/hydrophobicity converting material from the hydrophilicity/hydrophobicity converting material, thereby feeding the hydrophilic or hydrophobic feed material to a cell.

In order to attain the desire described above, according to an embodiment of the present disclosure, there is provided a cell culturing device including: an electrode on a surface of which a hydrophilicity/hydrophobicity converting material adapted to be electrically changed between hydrophilicity and hydrophobicity is provided, the electrode being disposed within a region in which a cell to be cultured is adapted to be accommodated.

According to another embodiment of the present disclosure, there is provided a cell culturing system including: a cell culturing device provided with an electrode on a surface of which a hydrophilicity/hydrophobicity converting material adapted to be electrically changed between hydrophilicity and hydrophobicity is provided, the electrode being disposed within a region in which a cell to be cultured is adapted to be accommodated; and a power source applying a voltage to the electrode.

According to still another embodiment of the present disclosure, there is provided a cell culturing method including: applying a voltage to an electrode on a surface of which a hydrophilicity/hydrophobicity converting material adapted to be electrically changed between hydrophilicity and hydrophobicity is provided within a region in which a cell to be cultured is adapted to be accommodated, thereby changing the hydrophilicity/hydrophobicity of the hydrophilicity/hydrophobicity converting material; desorbing a hydrophilic or hydrophobic feed material adsorbed to the hydrophilicity/hydrophobicity converting material in accordance with the change in the hydrophilicity/hydrophobicity of the hydrophilicity/hydrophobicity converting material; and feeding the hydrophilic or hydrophobic feed material thus desorbed to the cultured cell.

As set forth hereinabove, according to the present disclosure, the novel technique is provided in the cell culturing. That is to say, it is possible to provide the cell culturing device which is capable of controlling a feed time and a feed amount of feed material to be added to the culture solution in the cell culture, the cell culturing system including the same, and the cell culturing method using one of them.

### BRIEF DESCRIPTION OF THE DRAWINGS

FIG. 1 is a view schematically showing a cell culturing device according to a first embodiment of the present disclosure;

FIG. 2 is a view schematically showing a process for desorbing a feed material from a hydrophilicity/hydrophobicity converting material in the cell culturing device according to the first embodiment of the present disclosure;

FIG. 3 is a view schematically showing synthesis of N-isopropylacrylamido-vinylferrocene copolymer in the cell culturing device according to the first embodiment of the present disclosure;

FIG. 4 is a graph of a cyclic voltammogram, in a phase of application of a voltage, of N-isopropylacrylamido-vinylferrocene copolymer in the cell culturing device according to the first embodiment of the present disclosure;

FIG. 5 is a view, partly in block, schematically showing a cell culturing system according to a second embodiment of the present disclosure; and

FIG. 6 is a view, partly in block, schematically showing observation instrument according to Example of the first embodiment.

### DETAILED DESCRIPTION OF THE PREFERRED EMBODIMENTS

Embodiments of the present disclosure will be described in detail hereinafter with reference to the accompanying drawings. It is noted that embodiments which will be described below show examples of typical embodiments of the present disclosure, and thus the scope of the present disclosure is not intended to be construed in a limiting sense by the embodiments disclosed herein. The description will be given below in accordance with the following order.

1. First Embodiment: Cell Culturing Device

1-1. Configuration of Cell Culturing Device

1-2. Electrode

1-3. Hydrophilicity/Hydrophobicity Converting Material

1-4. Feed Material

2. Second Embodiment: Cell Culturing System

3. Third Embodiment: Cell Culturing Method

3-1. Cell Culturing Method

3-2. Cell and Culture Solution

4. Example

4-1. Configuration of Observation Instrument

4-2. Results of Observation

### 1. First Embodiment

### Cell Culturing Device

Hereinafter, a cell culturing device according to a first embodiment of the present disclosure will be described in detail.

### 1-1. Configuration of Cell Culturing Device

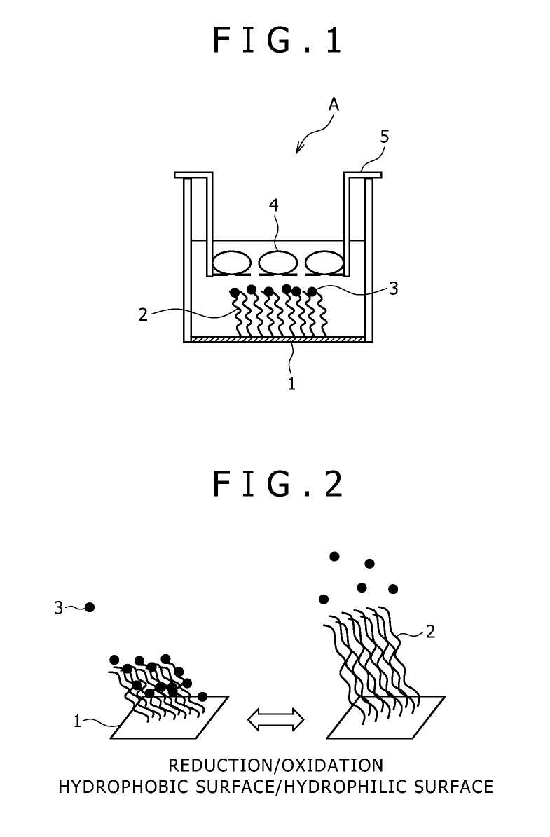

FIG. 1 is a schematic view of the cell culturing device according to the first embodiment of the present disclosure. The cell culturing device A includes an electrode 1 and a hydrophilicity/hydrophobicity converting material 2. In this case, the electrode 1 is disposed within a region in which cells to be cultured are accumulated. Also, the hydrophilicity/hydrophobicity converting material 2 is provided on the electrode 1 and can be electrically changed between hydrophilicity and hydrophobicity. A feed material 3 which is to be fed to each of the cells 4 is adsorbed to the hydrophilicity/hydrophobicity converting material 2.

In the cell culturing device A, for controlling contact between the feed materials 3 and the cells 4 only through the electrical hydrophilicity/hydrophobicity conversion of the hydrophilicity/hydrophobicity converting material 2, it is only necessary to hold a state in which each of the cells 4, and the electrode 1 to which the feed materials 3 are adsorbed do not come in contact with each other. For this reason, each of the cells 4 may be insulated from the electrode 1 through a diaphragm or the like. On the other hand, the diaphragm described above needs to have permeability for the feed materials 3 which are to be fed to the cells 4. For this reason, in the cell culturing device A, a diaphragm made of a filter member or the like is preferable. For example, in FIG. 1, an insert 5 is exemplified as the diaphragm. Use of the insert 5 results in that the diaphragm is provided in a horizontal direction with respect to a bonding surface of each of the cells 4, and thus each of the cells 4, and the electrode 1 can be partitioned in a vertical direction through the diaphragm. In addition, the diaphragm may also be provided in the vertical direction with respect to the bonding surface of each of the cells 4, and thus each of the cells 4, and the electrode 1 may also be partitioned in the horizontal direction through the diaphragm. Although in explaining the diaphragm, the cell 4 is supposed to be an adhesive cell, the cell 4 may also be a floating cell. Also, it is only necessary that the diaphragm is constructed so as to correspond to a property of each of the cells 4 in such a way that the state is held in which each of the cells 4, and the electrode 1 do not come into contact with each other. When the cell 4 is the adhesive cell, it is also possible to hold the state in which each of the cells 4, and the electrode 1 do not come into contact with each other without providing the diaphragm. It is not necessary to adopt a configuration in which the electrode 1 is disposed in a position, where the electrode 1 can contact the culture solution, located in an upper portion within the region, such as a flask for cell culture, in which the cells 4 are accommodated. In addition, a configuration is also preferable in which the insert 5 is used, and plural electrodes 1 are disposed in both of the upper portion and a low portion within the region in which the cells 4 are accommodated.

### 1-2. Electrode

The electrode 1 provided in the cell culturing device A may be made of any of materials as long as such a material is a conductive material in which deterioration such as corrosion is not caused within the cell culturing solution and which has high biocompatibility. For example, a carbon electrode, conductive polymer or the like are preferable as the material for the electrode 1 in addition to a metallic material such as gold, platinum, an iridium oxide or a titanium nitride.

### 1-3. Hydrophilicity/Hydrophobicity Converting Material

FIG. 2 is a view showing a process for desorbing the feed material from the hydrophilicity/hydrophobicity converting material in the cell culturing device according to the first embodiment of the present disclosure.

The hydrophilicity/hydrophobicity converting materials 2 are disposed on a surface of the electrode 1. In the hydrophilicity/hydrophobicity converting material 2 which can be electrically changed in the hydrophilicity/hydrophobicity, the application of a variable voltage results in the hydrophilicity/hydrophobicity being changed. As a result, the feed material 3 which has been previously adsorbed to the hydrophilicity/hydrophobicity converting material 2 loses the affinity with the hydrophilicity/hydrophobicity converting material 2 to be discharged into the cell culturing solution, thereby being fed to the cell 4.

FIG. 2 exemplifies a process in which the hydrophilicity/hydrophobicity converting material 2 which has shown the hydrophobicity before application of the suitable voltage to the electrode 1 comes to show the hydrophilicity by being electrically oxidized, and the feed material 3 which has been adsorbed to the hydrophilicity/hydrophobicity converting material 2 and which has shown the hydrophobicity loses the affinity with the hydrophilicity/hydrophobicity converting material 2 to be discharged into the cell culturing solution. Contrary to this, a process may also be adopted in which the hydrophilicity/hydrophobicity converting material 2 has shown the hydrophilicity before application of the suitable voltage to the electrode 1 comes to show the hydrophobicity by being electrically reduced, and the feed material 3 which has been adsorbed to the hydrophilicity/hydrophobicity converting material 2 and which has shown the hydrophilicity loses the affinity with the hydrophilicity/hydrophobicity converting material 2 to be discharged into the cell culturing solution.

In the cell culturing device A according to the first embodiment of the present disclosure, the application of the suitable voltage results in the affinity between the hydrophilicity/hydrophobicity converting material 2 and the feed material 3 being changed. As a result, the feed material 3 is discharged into the cell culturing solution to be fed to the cell 4. The hydrophilicity/hydrophobicity conversion of the hydrophilicity/hydrophobicity converting material 2 may be available in any pattern from the hydrophobicity to the hydrophilicity or from the hydrophilicity to the hydrophobicity.

The hydrophilicity/hydrophobicity converting material 2 disposed on the electrode 1 is composed of a material having a property of responding to an electrical change to change the hydrophilicity/hydrophobicity. More preferably, the hydrophilicity/hydrophobicity converting material 2 is polymer composed of a hydrophilicity/hydrophobicity converting unit and an electron accepting unit. A redox state in terms of the entire polymer in the polymer composed of the hydrophilicity/hydrophobicity converting unit and the electron accepting unit through giving and receiving of the electrons in the electron accepting unit after application of the suitable voltage to the electrode 1. The change in the redox in the polymer described above causes a change in the property of the hydrophilicity or the hydrophobicity of the hydrophilicity/hydrophobicity converting unit. As a result, it becomes possible to cause a change in the hydrophilicity/hydrophobicity of the polymer described above through the application of the suitable voltage to the electrode 1. As for a form of coupling between the hydrophilicity/hydrophobicity converting unit and the electron accepting unit in the polymer concerned, any form typified by a form in which the electron accepting unit is coupled as a component of copolymer, and a form in which the electron accepting unit is coupled to a side chain may be adopted.

The hydrophilicity/hydrophobicity converting unit features polymer composed of a material having a hydrophilic group and a hydrophobic group in a side chain thereof. When the oxidation of the polymer described above proceeds by the redox reaction through the electron accepting unit, the unit which has shown the hydrophobicity becomes hydrophilic due to ionization of the unit itself, an influence of paired ions in the vicinity of the unit itself, or the like. On the other hand, when the reduction of the polymer described above proceeds, the unit which has shown the hydrophilicity forms a structure of an aggregate to become hydrophobic.

The hydrophilicity/hydrophobicity converting unit described above, for example, includes polymer composed of such as a (meta)acrylamide compound, a N-alkyl substituted (meta)acrylamide derivative, an N,N dialkyl substituted (meta)acrylamide derivative, a (meta)acrylamide derivative having a cyclic group, and a vinyl ether derivative. More specifically, the hydrophilicity/hydrophobicity converting unit described above is polymer composed of acrylamide, methacrylamide, N-ethylacrylamide, N-n-propylacrylamide, N-n-propylmethacrylamide, N-isopropylacrylamide, N-isopropylmethacrylamide, N-cyclopropylacrylamide, N-cyclopropylmethacrylamide, N-ethoxyethylacrylamide, N-ethoxyethylmethacrylamide, N-tetrahydrofurfurylacrylamide, N-tetrahydrofurfurylmethacrylamide, N,N-dimethyl(meta-) acrylamide, N,N-ethylmethylacrylamide, N—N-diethylacrylamide, 1-(1-oxo-2-propenyl)-pyrrolidine, 1-(1-oxo-2-propenyl)-piperidine, 4-(1-oxo-2-propenyl)-morphorine, 1-(1-oxo-2-methyl-2-propenyl)-pyrrolidine, 1-(1-oxo-2-methyl-2-propenyl)-piperidine, 4-(1-oxo-2-methyl-2-propenyl)-morphorine, methylvinylether or the like. As for hydrophilicity/hydrophobicity converting unit concerned, a polymer composed of the above-described monomers and the other monomers, or a mixture of polymer and copolymer may also be used in addition to the polymer composed of one of the above-described monomers.

The feature of the electron accepting unit is to have the stable redox characteristics. As a result, the redox state of the electrode becomes reversible. The electron accepting unit, for example, includes a conjugated system molecule such as thiophene or aniline, an organic metallic complex, and an organic radical molecule.

As for the organic metallic complex concerned, metallocene is preferably used. More specifically, it is possible to use ferrocene, titanocene, vanadocene, chromocene, manganocene, cobaltocene, nickelocene, zirconocene, ruthenocene, osmocene, and the like.

N-isopropylacrylamido-vinylferrocene copolymer is preferable for the hydrophilicity/hydrophobicity converting material composed of the hydrophilicity/hydrophobicity converting unit and electron accepting unit described above.

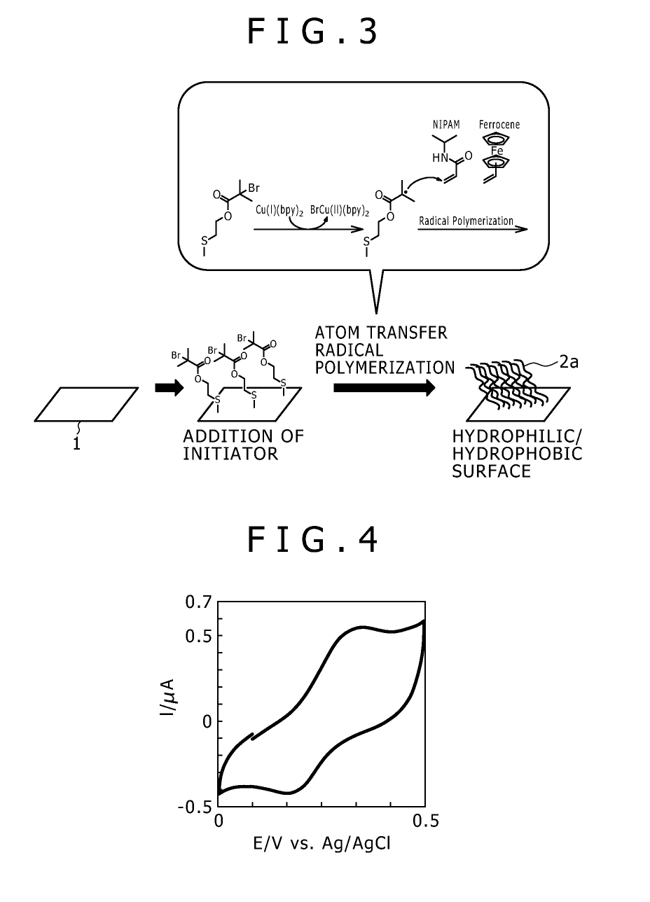

FIG. 3 shows a polymerization process in the electrode 1 by exemplifying N-isopropylacrylamido-vinylferrocene. N-isopropylacrylamido and vinylferrocene are used in the hydrophilicity/hydrophobicity converting unit and the electron accepting unit, respectively. Also, gold is used in the electrode 1. Bis[2-(2′-bromoisobutyryloxy)ethyl]disulfide is used as an initiator.

Firstly, a thiol group of the initiator is coordination-bonded to the surface of the electrode 1. Next, N-isopropylacrylamido and vinylferrocene are added to the surface of the electrode 1, and a copper(I)bipyridine complex as a catalyst is added thereto. Also atom transfer radical polymerization is carried out, thereby providing polymer 2a composed of N-isopropylacrylamido-vinylferrocene copolymer on the surface of the electrode 1.

In FIG. 3, as for the disposition of the polymer 2a on the electrode 1, a method of carrying out the polymerization on the surface of the electrode 1 is shown. However, a method may also be adopted in which the polymer 2a after completion of the synthesis is dissolved or dispersed into a suitable solvent, thereby coating the electrode 1 with the polymer 2a thus dissolved or disposed. Any of the methods described above is possible as a method of disposing the polymer 2a on the substrate 1.

FIG. 4 is a cyclic voltammogram in a phase of application of the suitable voltage in the N-isopropylacrylamido-vinylferrocene copolymer described above. As shown in the cyclic voltammogram concerned, the N-isopropylacrylamido-vinylferrocene copolymer disposed on the surface of the electrode 1 changes a redox state by application of the suitable voltage to the electrode 1. In addition, the using of the cyclic voltammogram or the like shown in FIG. 4 makes it possible to monitor the redox state on the electrode 1.

The change in the redox state on the electrode 1, and the change in the hydrophilicity/hydrophobicity of the polymer 2a show a correlative relationship. For this reason, the control for a feed amount of feed materials 3 which are discharged from the polymer 2a can be carried out through the change in the redox state of the polymer 2a in accordance with the control for the suitable voltage applied to the electrode 1. From this, with the cell culturing device A according to the first embodiment of the present disclosure, the feed of the feed materials 3 to the cells 4 can be carried out with high precision and reproducibility.

### 1-4. Feed Material

The feed material 3 which is desorbed from the hydrophilicity/hydrophobicity converting material 2 disposed on the surface of the electrode 1 to be fed to the cell 4 may be a material showing the affinity of any of the hydrophilicity or the hydrophobicity. The hydrophilicity or the hydrophobicity of the hydrophilicity/hydrophobicity converting material 2 can be previously determined depending on the property of the feed material 3. The feed material 3, for example, may be any of a protein such as cytokine, hormone or an antibody, a nucleic acid such as a vector or a nucleic-acid aptamer, a lipid, a suger chain, a low-molecular compound, or the like as long as such a material is a material which is fed to the cell 4 in the cell culture.

By adopting the constitution described above, with the cell culturing device according to the first embodiment of the present disclosure, the application of the suitable voltage to the electrode 1 changes the hydrophilicity/the hydrophobicity of the hydrophilicity/hydrophobicity converting material 2 disposed on the surface of the electrode 1. Thus, the affinity between the feed material 3 adsorbed to the hydrophilicity/hydrophobicity converting material 2 and the hydrophilicity/hydrophobicity converting material 2 is lost to discharge the feed material 3 into the cell culturing solution, thereby making it possible to feed the feed material 3 to the cell 4.

### 2. Second Embodiment

### Cell Culturing System

Next, a description will be given with respect to a cell culturing system according to a second embodiment of the present disclosure.

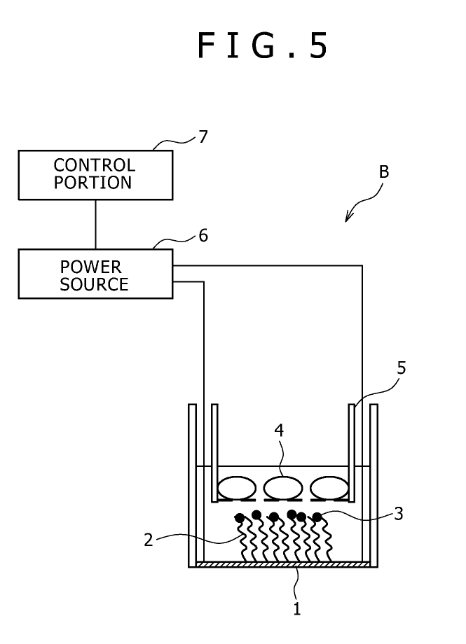

The cell culturing system B shown in FIG. 5 includes a power source 6 for applying a suitable voltage to the electrode 1 in addition to the components of the cell culturing device A shown in FIG. 1. Since the configuration of the cell culturing device A is the same as that described with reference to FIGS. 1 to 4, a repeated description is omitted here for the sake of simplicity. The cell culturing system B may include a control portion 7 for controlling either an application time or an application amount of voltage in the power source 6. The control portion 7 either may have a configuration of being included in the power source 6, or may have a configuration of being provided separately from the power source 6 in the manner as shown in FIG. 5. In addition, the cell culturing system B may have a configuration in which plural cell culturing devices A are connected to one power source 6.

By adopting the configuration described above, with the cell culturing system B, the suitable voltage can be applied to the electrode 1 by the power source 6 to change the hydrophilicity/the hydrophobicity of the hydrophilicity/hydrophobicity converting material 2 disposed on the surface of the electrode 1. Thus, the affinity between the feed material 3 adsorbed to the hydrophilicity/hydrophobicity converting material 2 and the hydrophilicity/hydrophobicity converting material 2 can be lost to discharge the feed material 3 into the cell culturing solution, thereby making it possible to feed the feed material 3 to the cell 4. In addition thereto, the cell culturing system B includes the control portion 7, thereby making it possible to control the time at which the suitable voltage is applied to the electrode 1 by the power source 6 or the application amount of the voltage, and to control either the application time or the feed amount of feed materials 3 to the cells 4.

### 3. Third Embodiment

### Cell Controlling Method

Next, a description will be given with respect to a cell culturing method according to a third embodiment of the present disclosure.

### 3-1. Cell Culturing Method

The cell culturing method according to the third embodiment of the present disclosure includes: a procedure for electrically changing the hydrophilicity/hydrophobicity of the hydrophilicity/hydrophobicity converting material by applying the suitable voltage to the electrode which is disposed within the region capable of accommodating therein the cell to be cultured and on the surface of which the hydrophilicity/hydrophobicity converting material whose hydrophilicity/hydrophobicity can be electrically changed is provided; a procedure for desorbing the hydrophilic or hydrophobic feed material adsorbed to the hydrophilicity/hydrophobicity converting material by the change in the hydrophilicity/hydrophobicity of the hydrophilicity/hydrophobicity converting material; and a procedure for feeding the feed material to the cell to be calculated. That is to say, the cell culturing method according to the third embodiment is the cell culturing method using the cell culturing device A according to the first embodiment. It is noted that although in the third embodiment of the present disclosure, the cell culturing method is described as being composed of the three procedures, the two procedures after the procedure for applying the suitable voltage to the electrode 1 of the cell culturing device A has been carried out are continuously generated and thus can also be grasped as one procedure.

In addition to the configuration shown in FIG. 1, the cell culturing device A, for example, can also adopt a configuration in which plural electrodes 1 are provided together with the insert 5 within the region capable of accommodating therein the cells 4, and a concentration gradient of the feed material 3 is formed within the cell culturing solution. By adopting such a configuration, the cell 4 can also be cultured in a state of having a polarity.

In addition thereto, the cell culturing method of the third embodiment further includes a procedure for desorbing the feed material adsorbed to the hydrophilicity/hydrophobicity converting material at an arbitrary time or amount from the electrode by controlling an application amount or application time of the voltage. That is to say, in this case, the cell culturing method of the third embodiment is a cell culturing method using the cell culturing system B of the second embodiment.

In the cell culturing method of the third embodiment, the cell culturing method having both of the high precision and reproducibility becomes possible by electrically controlling the addition amount or addition time of the feed material which is to be fed to the cell. In addition thereto, even when plural cell culturing devices are used in the cell culturing system of the third embodiment, thereby carrying out plural kinds of cell cultures at the same time, the cell culturing method having both of the high precision and reproducibility can be carried out.

### 3-2. Cell and Culture Solution

The cell which is cultured in the cell culturing device of the first embodiment is by no means limited, and thus may be any of a plant cell and an animal cell. With regard to the cell culturing solution as well, it is possible to use the culture solution suitable for the cell described above.

For example, in the culture of a stem cell such as an embryo-derived stem cell, an induced pluripotent stem cell or a mesenchymal stem cell, control for a differentiated state is necessary in a phase of the culture. In recent years, the investigation for controlling differentiation by using a culture solution or additive whose composition is chemically obvious has been progressed. An efficient method having high reproducibility is required for addition of a material for control for the differentiation to the cell. Thus, the cell culturing method according to the third embodiment of the present disclosure is suitable for culture of the stem cells described above.

It is noted that the present disclosure can also adopt the following constitutions.

(1) A cell culturing device including: an electrode on a surface of which a hydrophilicity/hydrophobicity converting material adapted to be electrically changed between hydrophilicity and hydrophobicity is provided, the electrode being disposed within a region in which a cell to be cultured is adapted to be accommodated.

(2) The cell culturing device described in the paragraph (1), in which the hydrophilicity/hydrophobicity converting material is polymer composed of a hydrophilicity/hydrophobicity converting unit and an electron accepting unit.

(3) The cell culturing device described in the paragraph (1), in which the hydrophilicity/hydrophobicity converting material is N-isoprophyacrylamide-vinylferrocene copolymer.

(4) The cell culturing device described in any one of the paragraphs (1) to (3), in which a hydrophilic or hydrophobic feed material which is to be fed to a cell is adsorbed to the hydrophilicity/hydrophobicity converting material.

(5) The cell culturing device described in the paragraphs (4), in which by applying a voltage to the electrode to change the hydrophilicity/hydrophobicity of the hydrophilicity/hydrophobicity converting material, the feed material is desorbed from the hydrophilicity/hydrophobicity converting material to be fed to the cell to be cultured.

(6) The cell culturing device described in the paragraphs (5), further including a diaphragm isolating the cell to be cultured in a state in which the cell to be cultured does not contact the electrode, and adapted to cause the feed material desorbed from the hydrophilicity/hydrophobicity converting material to reach the cell to be cultured in the region.

(7) A cell culturing system including: a cell culturing device provided with an electrode on a surface of which a hydrophilicity/hydrophobicity converting material adapted to be electrically changed between hydrophilicity and hydrophobicity is provided, the electrode being disposed within a region in which a cell to be cultured is adapted to be accommodated; and a power source applying a voltage to the electrode.

(8) The cell culturing system described in the paragraphs (7), further including a control portion configured to control an application amount or application time of the voltage in the power source.

(9) A cell culturing method including: applying a voltage to an electrode on a surface of which a hydrophilicity/hydrophobicity converting material adapted to be electrically changed between hydrophilicity and hydrophobicity is provided within a region in which a cell to be cultured is adapted to be accommodated, thereby changing the hydrophilicity/hydrophobicity of the hydrophilicity/hydrophobicity converting material; desorbing a hydrophilic or hydrophobic feed material adsorbed to the hydrophilicity/hydrophobicity converting material in accordance with the change in the hydrophilicity/hydrophobicity of the hydrophilicity/hydrophobicity converting material; and feeding the hydrophilic or hydrophobic feed material thus desorbed to the cultured cell.

(10) The cell culturing method described in the paragraph (9), further including: desorbing the feed material adsorbed to the hydrophilicity/hydrophobicity converting material at an arbitrary time or at an arbitrary amount from the hydrophilicity/hydrophobicity converting material by controlling an application amount or application time of the voltage.

### 4. Example

A behavior of the feed material caused by the change in the hydrophilicity/hydrophobicity of the surface of the electrode in the cell culturing device according to the first embodiment or in a cell culturing system according to the second embodiment was observed by using observation instrument.

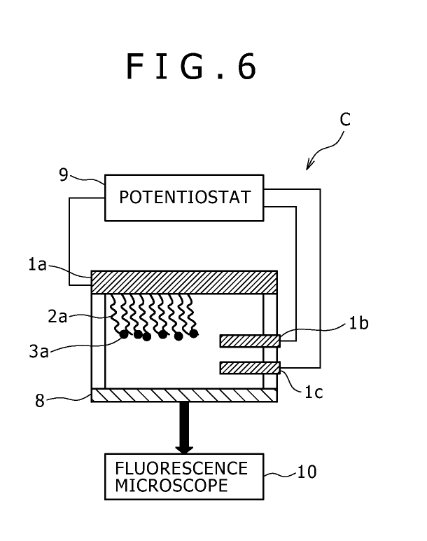

4-1. Configuration of Observation Instrument FIG. 6 shows a configuration of the observation instrument C used in Example. The observation instrument C includes three kinds of electrodes: a working electrode 1a; a counter electrode 1b; and a reference electrode 1c, a potentiostat 9, and a fluorescence microscope 10. In this case, the potentiostat 9 controls the three kinds of electrodes 1a, 1b, and 1c. Also, the fluorescence microscope 10 is used to observe changes of the surfaces of the three kinds of electrodes 1a, 1b, and 1c. Polymer 2a to which a fluorescence lipid 3a is adsorbed is provided on a surface of the working electrode 1a. For the purpose of observing the behavior of the fluorescence lipid 3a after application of the suitable voltage, a glass plate 8 is provided between the working electrode 1a and the fluorescence microscope 10. In addition, a space which contain therein the polymer 2a and which is held between the working electrode 1a and the glass plate 8 is filled with a phosphate buffered saline.

The working electrode la was made of gold. N-isopropylacrylamide-vinylferrocene copolymer was used in the polymer 2a, and the synthesis and the disposition of N-isopropylacrylamide-vinylferrocene copolymer to the working electrode 1a were carried out by utilizing the same method described in 1-3. After completion of the synthesis, the working electrode 1a having the polymer 2a disposed thereon was made to show the hydrophobic state by application of the suitable voltage. Boron-Dipyrromethene (BODIPY) was used as the fluorescence lipid 3a. The BODIPY was dissolved into ethanol (at a rate of 1 μg/1 ml), and was then applied to the surface of the working electrode 1a having the polymer 2a disposed thereon several times.

### 4-2. Results of Observation

The behavior of the fluorescence lipid 3a after application of the suitable voltage to the working electrode 1a is observed by using the fluorescence microscope 10 of the observation instrument C. Ten minutes after the suitable voltage of 0.5 V (vs. Ag/AgCl) was applied to the working electrode 1a, a signal derived from the fluorescence lipid 3a on the working electrode 1a was weakened. From this, it was thought that the fluorescence lipid 3a adsorbed to the polymer 2a was desorbed from the polymer 2a to diffuse into the phosphate buffered saline.

The results of the observation in Example show that in the cell culturing device or the cell culturing system according respectively to the first embodiment and second embodiment of the present disclosure, the hydrophilicity/hydrophobicity of the hydrophilicity/hydrophobicity converting material disposed on the surface of the electrode is changed through the application of the suitable voltage to the electrode and thus the feed material adsorbed to the hydrophilicity/hydrophobicity converting material can be desorbed from the hydrophilicity/hydrophobicity converting material to be fed to the cell to be cultured.

In the cell culturing device, the cell culturing system or the cell culturing method according to the first, second or third embodiment of the present disclosure, the feed material can be discharged into the cell culturing solution with high precision to be fed to the cells. In particular, the cell culturing device according to the first embodiment of the present disclosure is suitably used for supply of the material used to control the differentiation in the stem cell to the stem cell.

The present technology contains subject matter related to that disclosed in Japanese Priority Patent Application JP 2011-169818 filed in the Japan Patent Office on Aug. 3, 2011, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A cell culturing device, comprising:
an electrode on a surface of which a hydrophilicity/hydrophobicity converting material adapted to be electrically changed between hydrophilicity and hydrophobicity is provided, said electrode being disposed within a region in which a cell to be cultured is adapted to be accommodated.

2. The cell culturing device according to claim 1, wherein said hydrophilicity/hydrophobicity converting material is polymer composed of a hydrophilicity/hydrophobicity converting unit and an electron accepting unit.

3. The cell culturing device according to claim 1, wherein said hydrophilicity/hydrophobicity converting material is N-isopropylacrylamide-vinylferrocene copolymer.

4. The cell culturing device according to claim 1, wherein a hydrophilic or hydrophobic feed material which is to be fed to a cell is adsorbed to said hydrophilicity/hydrophobicity converting material.

5. The cell culturing device according to claim 4, wherein by applying a voltage to said electrode to change the hydrophilicity/hydrophobicity of said hydrophilicity/hydrophobicity converting material, said feed material is desorbed from said hydrophilicity/hydrophobicity converting material to be fed to said cell to be cultured.

6. The cell culturing device according to claim 5, further comprising:
a diaphragm isolating said cell to be cultured in a state in which said cell to be cultured does not contact said electrode, and adapted to cause said feed material desorbed from said hydrophilicity/hydrophobicity converting material to reach said cell to be cultured in said region.

7. A cell culturing system, comprising:
a cell culturing device provided with an electrode on a surface of which a hydrophilicity/hydrophobicity converting material adapted to be electrically changed between hydrophilicity and hydrophobicity is provided, said electrode being disposed within a region in which a cell to be cultured is adapted to be accommodated; and
a power source applying a voltage to said electrode.

8. The cell culturing system according to claim 7, further comprising
a control portion configured to control an application amount or application time of the voltage in said power source.

9. A cell culturing method, comprising:
applying a voltage to an electrode on a surface of which a hydrophilicity/hydrophobicity converting material adapted to be electrically changed between hydrophilicity and hydrophobicity is provided within a region in which a cell to be cultured is adapted to be accommodated, thereby changing the hydrophilicity/hydrophobicity of said hydrophilicity/hydrophobicity converting material;
desorbing a hydrophilic or hydrophobic feed material adsorbed to said hydrophilicity/hydrophobicity converting material in accordance with the change in the hydrophilicity/hydrophobicity of said hydrophilicity/hydrophobicity converting material; and
feeding said hydrophilic or hydrophobic feed material thus desorbed to said cultured cell.

10. The cell culturing method according to claim 9, further comprising:
desorbing said feed material adsorbed to said hydrophilicity/hydrophobicity converting material at an arbitrary time or at an arbitrary amount from said hydrophilicity/hydrophobicity converting material by controlling an application amount or application time of the voltage.
